(19) United States
(12) Patent Application Publication
(10) Pub. No.: US 2010/0130461 A1
(43) Pub. Date: May 27, 2010

(54) Pharmaceutical composition for topical application

(76) Inventor: Bruce Burkenstock, SR., Destrehan, LA (US)

Correspondence Address: KEATY LAW FIRM, LLC, 365 CANAL STREET, Suite 2410, NEW ORLEANS, LA, 70130, US

(21) Appl. No.: 12/657,663

(22) Filed: Jan. 26, 2010

Related U.S. Application Data
(62) Division of application No. 11/904,461, filed on Sep. 27, 2007.

(51) Int. Cl. A61K 31/573; A61P 17/00

(52) U.S. Cl. 514/181

(57) ABSTRACT

A topical composition for treatment of contact dermatitis comprising a corticosteroid, one or more drying agents, such as zinc oxide and talc, an alcohol solvent, a humectant and a viscosity agent, such as bentonite. The humectant may be glycerin, and the carrier is water.

### CROSS-REFERENCE TO RELATED APPLICATIONS

This application is a divisional application of my co-pending application Ser. No. 11/904,461 filed on Sep. 27, 2007 entitled “Pharmaceutical Composition for Topical Applications,” the full disclosure of which is incorporated by reference herein, and priority of which is hereby claimed.

### BACKGROUND OF THE INVENTION

This invention relates to a pharmaceutical composition for topical application onto skin, and, more particularly to a pharmaceutical composition that can be utilized for the treatment of skin disorders resulting from inflammation of the skin.

Most of the inflammatory skin diseases or disorders are caused by inflammatory agents, such as, for instance bacterium, fungi, virus, allergens, hormonal imbalance and the like. Some of the most common skin diseases or disorders include contact dermatitis caused by an allergic or irritant reaction (such as that caused by poison ivy, poison oak or poison sumac, or other forms of allergic or irritant contact dermatitis). Skin disorders may be also caused by infection, irritation or aggravation of another condition such as occurs with acne, and other rashes, dermatoses or skin eruptions.

Irritants can also cause skin dryness, pruritus (itch) secondary to inflammation, and pain due to fissures, blisters, and ulcers. Mild irritants produce erythema, microvesiculation, and oozing that may be indistinguishable from allergic contact dermatitis. Strong irritants, such as poison ivy or insect bites cause blistering, erosion, and ulcers of the skin. Allergic contact dermatitis, in its mild form, is similar in appearance to the irritant eruption.

The inflammatory skin disorders resulting from insect bites, similar to the contact dermatitis and acne, cause itching and discomfort, as well as swelling, which is exacerbated when a person tries to relieve the itching by scratching the inflamed area, with a potential for causing damage to the underlying layers of dermis. Moreover, a damaged outer skin layer may lead a bacterial infection.

The pharmaceutical sciences created various creams, ointments and lotions for treating skin diseases and disorders. Some of these creams contain antifungal agents, steroidal agents and/or antibacterial agents. Topical corticosteroids, such as hydrocortisone, usually treat contact dermatitis, but they do not prevent new vesicles from forming or dry up oozing, weeping patches and vesicles rapidly. Soothing lotions and drying agents are shown effective in drying oozing, weeping patches, vesicles and erosions.

The calamine lotion is often used as an antipruritic composition to treat sunburn, ezcema, poison ivy, insect bites and stings. Calamine is a mixture of zinc oxide (ZnO) with about 0.5% ferric (III) oxide (Fe₂O₃). It is also used as a mild antiseptic to prevent infections that can be caused by scratching the affected area. However, in 1992 the U.S. FDA (Food and Drug Administration) advised that there was no proof the main ingredients in calamine (zinc oxide and ferric oxide) had any real therapeutic effect on rashes and itching. When used alone, calamine lotion usually does not provide relief from the inflammation caused by the dermatitis.

Many antifungal, analgesic, anti-allergic, anti-inflammatory topical compositions use petroleum or oil-based substances as a carrier. However, many users report that petroleum-based lotions and creams tend to leave an oily residue and result in greasy feeling following their application to the skin. Therefore, instead of drying up the oozing skin erosions they tend to inhibit drying and may not be desirable in certain applications.

The present invention contemplates elimination of drawbacks associated with the conventional anti-inflammatory, anti-itch topical compositions and provision of a pharmaceutical composition that can reduce redness, swelling, inflammation, pruritus and oozing without leaving a greasy layer on the skin surface.

### SUMMARY OF THE INVENTION

It is, therefore, an object of the present invention to provide a pharmaceutical composition that can be used for topical application in treating inflammatory skin disorders.

It is another object of the invention to provide a topical composition that does not leave an oily residue upon application.

It is a further object of the present invention to provide a method of treating contact dermatitis with a corticosteroid-containing composition that facilitates healing time, while reducing itching and swelling.

These and other objects of the invention are achieved through a provision of a topical composition, which comprises one or more drying agents, a corticosteroid in the amount of between about 0.75 and 1.26% by weight, a solvent, a humectant and a viscosity-building agent mixed with a pharmaceutically-acceptable carrier.

In the preferred embodiments, the composition of the present invention comprises, by weight, between about 10 and 18.2 percent of a water-insoluble topical protectant, or drying agent, such as zinc oxide, an equal amount of an astringent powder, such as talcum powder, between about 22 and 37.26 percent of a solvent, for instance a monohydric alkanol, between about 9 and 15.46 percent of a humectant agent, such as glycerin, between about 0.75 and 1.26 percent of a synthetic corticosteroid, such as hydrocortisone, and between about 1 and 20.06 percent of viscosity-building agent, such as bentonite mixed with a pharmaceutically or cosmetically acceptable carrier to form a cream or lotion.

The pharmaceutically acceptable carrier can be water, or distilled water. The solvent may be isopropyl alcohol. The composition may contain one or more drying agents. In the preferred embodiment, the drying agents are zinc oxide and talc. The composition may also contain a trace amount of a coloring agent.

The method of the present invention provides for the topical application of the composition of this invention two to four times daily until the skin disorder is healed.

### DETAIL DESCRIPTION OF THE PREFERRED EMBODIMENT

As used herein, the term “pharmaceutical or cosmetic composition” is intended to describe compositions for topical application to human skin.

The term “skin” as used herein includes the skin on the face, neck, chest, back, arms, axilla, hands, legs, and scalp.

Except in the examples, or where otherwise explicitly indicated, all numbers in this description indicating amounts of material or conditions of reaction, physical properties of materials and/or use are to be understood as modified by the word “about.” All amounts are by weight of the composition, or percent by weight unless otherwise specified.

It should be noted that in specifying any range of concentration, any particular upper concentration can be associated with any particular lower concentration.

The term “comprising” is used herein in its ordinary meaning and means including, made up of, composed of, consisting and/or consisting essentially of. In other words, the term is defined as not being exhaustive of the steps, components, ingredients, or features to which it refers.

According to one aspect of the present invention there is provided a topical pharmaceutical or cosmetic composition comprising one or more drying agents, a corticosteroid, a solvent, a humectant and a viscosity-building agent mixed with water to form a lotion or cream.

In the preferred embodiments, the composition comprises, by weight, between about 10 and 18.2 percent of a water-insoluble topical protectant, or drying agent, such as zinc oxide, an equal amount of an astringent powder, such as talcum powder, between about 22 and 37.26 percent of a solvent, for instance a monohydric alkanol, between about 9 and 15.46 percent of a humectant agent, such as glycerin, between about 0.75 and 1.26 percent of a synthetic corticosteroid, such as hydrocortisone, and between about 1 and 20.06 percent of viscosity-building agent, such as bentonite mixed with a pharmaceutically or cosmetically acceptable carrier to form a cream or lotion.

In the preferred embodiments, the composition of the present invention comprises by weight, between 10.92 and 18.2 percent of zinc oxide, an equal amount of talcum powder, between 22.36 and 37.26 percent of a solvent, for instance isopropyl alcohol, between 9.28 and 15.46 percent of glycerin, between 0.75 and 1.26 percent of hydrocortisone, and between 1.25 and 20.06 percent of bentonite, mixed with water to form a cream or lotion. An optional colorant agent or perfume may be added to the composition. In the preferred embodiments, the coloring agent is added in the amount of less than 1 percent.

The most preferred embodiment is anticipated to comprise, by weight, about 14.56 percent of zinc oxide, an equal amount of talcum powder, about 29.81 percent of isopropyl alcohol, about 12.37 percent of glycerin, about 1 percent of hydrocortisone powder, and about 1.66 percent of bentonite, mixed with about 26.04 percent of water. A trace amount of a coloring agent may be added.

The cosmetically acceptable vehicle may act as a dilutent, dispersant or carrier for the skin benefit ingredients in the composition, so as to facilitate their distribution when the composition is applied to the skin.

The vehicle may be aqueous, anhydrous or an emulsion. Preferably, the composition is aqueous. Water will be in amounts, which may range from 19 to 33% by weight, preferably from 19.53 to 32.55%, optimally about 26.04% by weight.

Besides water, relatively volatile solvents may also serve as carriers within the compositions of the present invention. Most preferred are monohydric alkanols, and more specifically isopropyl alcohol (C₃H₈O). Other solvents may include ethyl alcohol and methyl alcohol.

The drying agent, and in particular zinc oxide, is designed to promote drying of moist areas of the skin and protection of the skin by forming a water-impermeable layer. The coating layer prevents skin drying and facilitates healing. Preferred drying agents include zinc-containing drying agents such as zinc oxide, zinc acetate, zinc stearate and zinc sulfate. Talcum powder also acts as a drying agent and hence the present invention provides for the incorporation of equal amounts of corticosteroid and talcum powder. Talc is a mineral composed of hydrated magnesium silicate with the chemical formula H₂Mg₃(SiO₃)₄.

The corticosteroid is selected from the group consisting of mild-potency corticosteroids, such as hydrocortisone butyrate, hydrocortisone propionate, and hydrocortisone valerate.

In addition to the chemical compound glycerol, chemical formula HOCH₂CH(OH)CH₂OH, other humectants of the polyhydric alcohol-type may also be employed as acceptable carriers in the composition of this invention. The humectant aids in increasing the effectiveness of the composition, particularly in reducing skin irritation and scaling. Typical polyhydric alcohols include polyalkylene glycols, such as alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably glycerol or glycerine. The amount of humectant may range anywhere from 9 to 16%, preferably between 9.28 and 15.46% by weight of the composition.

The pharmaceutical/cosmetic compositions within the scope of the invention were prepared. A base formulation was made by mixing zinc oxide, hydrocortisone, talc, bentonite, glycerin, and isopropyl alcohol with water. In one exemplary embodiment, about 64 g of bentonite magma was diluted in water and mixed with 475 ml glycerin and a pint (473 ml) of 70% isopropyl alcohol. The first mixture was shaken well. Then, talc powder (424 gm) was added along with about 473 ml of alcohol and again shaken well. Then about 454 gm of zinc oxide powder was added together with 473 ml of 70% isopropyl alcohol. This fourth mixture was well shaken. Then, the rest of the total amount of zinc oxide (105 gm) was added along with the remaining talc powder (135 gm) and hydrocortisone powder (about 38.4 gm for 1% lotion) and with sufficient quantity of 70% alcohol to 3840 ml total of lotion. This last mixture was also shaken well.

After mixing all ingredients, the mixture was transferred to a high-speed blender and processed for 30-60 seconds to form a homogenous substance in the form of lotion. The composition can be packaged in individual containers ready for dispensing.

If desired, the lotion can be deposited into a 4-ounce container and sufficient quantity of isopropyl alcohol added to make 120 ml of lotion. Optionally, about 0.012-0.02 mg coloring agent can be added to give the lotion a flesh-like color. The lotion should be shaken before dispensing or application.

The following examples of the composition, by weight, can be prepared:

In Example I, 698.75 gm of zinc oxide and equal amount of talc were mixed with 48 gm of hydrocortisone, 80 gm bentonite, 593.75 gm glycerin, 1430.73 gm of isopropyl alcohol and 1250 gm water to form a lotion. 0.02 gm of a coloring agent was added. Example II provides for the mixing of 419.25 gm zinc oxide and equal amount of talc with 28.80 gm of hydrocortisone, 48 gm bentonite, 356.25 gm glycerin, 858.438 gm of isopropyl alcohol and 750 gm water to form a lotion. In Example II, 0.012 gm coloring agent was added.

In Example III, 559 gm of zinc oxide and equal amount of talc were mixed with 559 gm of hydrocortisone, 64 gm bentonite, 475 gm glycerin, 1144.584 gm of isopropyl alcohol and 1000 gm water to form a lotion. 0.016 gm of a coloring agent was added.

The composition of the invention is applied topically to the affected skin area until it has healed. For example, for contact dermatitis a composition of Examples I, II, or III is preferably administered two to four times a day for a period of at least one day until the skin heals. Preliminary tests were conducted by applying the composition of the present invention to the skin. The tests demonstrated that the compositions of the present invention are effective in reducing inflammation, swelling, itching, pain associated with the skin inflammation, pruritus and oozing that resulted from contact dermatitis, acne, and insect bites. The tests subjects reported relief from pain within minutes of application to the affected skin area.

The use of compounds of the general formula above and compositions including same, delivers anti-inflammatory, anti-swelling benefits with potential reduced irritation, redness, pruritis and oozing. The present invention provides a pharmaceutical or cosmetic composition and method of skin treatment using the above composition admixed with an acceptable vehicle, such as water. The topical composition of the present invention can be used for topical treatment of rashes, dermatoses and skin eruptions, when applied to the skin. The composition contains zinc oxide, talc and a corticosteroid of the appropriate potency for the condition being treated, promoting rapid drying of affected areas and coating the skin with the drying agent for protection and healing.

The composition of the present invention may also be in the form of a solution, spray, cream, or gel. The preferred form of the composition depends upon the condition being treated and the desired therapeutic effect. For example, treatment of a moist, acutely inflamed rash is preferably treated with a lotion, whereas treatment of a chronic dry patch is often treated more effectively with a cream. It is envisioned that the composition may be stronger in a cream form or gel, although a lotion form was described above.

It should be understood that the specific forms of the invention herein illustrated and described are intended to be representative only. Changes, including but not limited to those suggested in this specification, may be made in the illustrated embodiments without departing from the clear teachings of the disclosure. Accordingly, reference should be made to the following appended claims in determining the full scope of the invention.

1-10. (canceled)

11. A method for treating contact dermatitis, the method comprising applying to the skin a composition comprising one or more drying agents, a corticosteroid in the amount of between about 0.75 and 1.26% by weight, a solvent, a humectant and a viscosity-building agent mixed with a pharmaceutically-acceptable carrier.

12. The method of claim 11, wherein the corticosteroid is selected from the group consisting of hydrocortisone butyrate, hydrocortisone propionate and hydrocortisone valerate.

13. The method of claim 11, wherein said at least one drying agent is zinc oxide and the ratio of zinc oxide is between about 10.92 and 18.20% by weight.

14. The method of claim 11, wherein the composition comprises at least two drying agents, one of the drying agents is zinc oxide, another is talc, and wherein zinc oxide and talc are present in substantially equal amounts of between about 10.92 and 18.20% by weight.

15. The method of claim 11, wherein said solvent is a monohydric alkanol selected from the group consisting of isopropyl alcohol, ethyl alcohol and methyl alcohol.

16. The method of claim 15, wherein the composition comprises between about 22.36 and 37.26% by weight of isopropyl alcohol.

17. The method of claim 11, wherein said humectant is a polyhydric alcohol selected from the group consisting of glycerol, propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof.

18. The method of claim 11, wherein said humectant is glycerol in the amount of between about 9.28 and 15.46% by weight.

19. The method of claim 11, wherein said viscosity-building agent is bentonite in the amount of between about 1.25 and 2.08% by weight.

20. The method of claim 1, wherein said pharmaceutically-acceptable carrier is water.

21. (canceled)

22. A method of treating a disease or disorder of a skin comprising the step of topically administrating thereto a pharmaceutical composition containing: between about 10 and 18.2 percent zinc oxide, an equal amount of talcum powder, between about 22 and 37.26 percent isopropyl alcohol, between about 9 and 15.46 percent glycerin, between about 0.75 and 1.26 percent a corticosteroid, and between about 1 and 20.06 percent bentonite mixed with a pharmaceutically-acceptable carrier to form a cream or lotion, and wherein the pharmaceutical composition is effective in the treatment of contact dermatitis.

23. The method of claim 22, wherein said corticosteroid is hydrocortisone.

24. The method of claim 22, wherein the pharmaceutically-acceptable carrier is water.
